(19) United States
(12) Patent Application Publication
(10) Pub. No.: US 2005/0267357 A1
(43) Pub. Date: Dec. 1, 2005

(54) fMRI system for detecting symptoms associated with Attention Deficit Hyperactivity Disorder

(76) Inventors: Stephen M. Rao, Shorewood, WI (US); Catherine L. Elsinger, Wauwatosa, WI (US)

Correspondence Address: John J. Horn, Patent Counsel;Neurognostics, Inc., Suite 309, 10437 Innovation Drive, Milwaukee, WI, 53226-4815, US

(21) Appl. No.: 11/179,167

(22) Filed: Jul. 12, 2005

Related U.S. Application Data
(63) Continuation-in-part of application No. 10/970,927, filed on Oct. 21, 2004.
(60) Provisional application No. 60/512,940, filed on Oct. 21, 2003.

(51) Int. Cl. A61B 5/05

(52) U.S. Cl. 600/411

(57) ABSTRACT

A system based on the use of fMRI techniques for use in detecting neurological abnormalities indicative of Attention Deficit Hyperactivity Disorder (ADHD), in determining the severity of ADHD and in gauging the efficacy of medications used in treating ADHD. The system includes the steps of activating a selected region of the brain which is known to be affected by ADHD using a working memory and sustained attention task such as an N-Back task and concurrently acquiring fMRI image data responsive to the task. The patient's task-active fMRI data is then compared to reference fMRI data derived from a database of task-active fMRI data acquired from healthy individuals and determining whether the patient has symptoms related to ADHD. The extent of the patient's ADHD related symptoms and the severity of the disorder may also be assessed. Additionally, patients who are affected by ADHD may be administered medications intended to address their symptoms and based on comparing the severity of the patient's symptoms on and off therapy, the efficiency of the medication may be gauged and a measure may be provided of how well individual patients respond to a given medication.

### RELATED APPLICATIONS

This application claims the benefit of U.S. patent application Ser. No. 10/970,927 filed Oct. 21, 2004 and U.S. patent application Ser. No. 10/971,289 filed Oct. 21, 2004 U.S. provisional patent application No. 60/512,940 filed Oct. 21, 2003, which are incorporated herein by reference in their entirety.

### FIELD OF THE INVENTION

The present invention relates to systems for use in detecting symptoms of neurological disorders and more specifically to the use of functional magnetic resonance imaging (fMRI) in detecting symptoms, determining severity and assessing therapeutic efficacy in cases of Attention Deficit Hyperactivity Disorder (ADHD).

### BACKGROUND OF THE INVENTION

Attention Deficit Hyperactivity Disorder (ADHD) is a common neurobehavioral childhood disorder characterized by developmentally inappropriate levels of inattention, hyperactivity, and impulsivity. Recent prospective and retrospective studies indicate that at least half of ADHD children continue to exhibit symptoms of ADHD into adulthood. ADHD may affect up to 8-10% of children (American Association of Pediatrics, 2000) and may persist into adolescence in up to 80% of cases. Prevalence of ADHD in adults is estimated to be 4-5%, thus affecting 9.4 million adults in the US. ADHD is characterized by developmentally inappropriate symptoms of inattention, impulsivity, and hyperactivity that impair normal functioning. The diagnosis of ADHD is associated with low academic achievement, poor school performance, retention in grade, school suspensions and expulsions, poor peer and family relations, conduct problems and delinquency, early substance abuse, driving accidents and speeding violations, and, in adults, impaired marital/social relationships and underemployment. Neuropsychological studies have identified a wide range of cognitive deficits on measures of response inhibition, working memory, sustained attention, timing perception/reproduction, and conceptual reasoning. The diagnostic criteria for ADHD published by the American Psychiatric Association (Diagnostic and Statistical Manual of Mental Disorders; DSM-IV; 1994) are the most widely used at present. Alternate criteria include the International Statistical Classification of Diseases and Related Health Problems (tenth revision; ICD-10; Swanson et al.; 1998), that define the diagnosis of hyperkinetic disorder. The ICD-10 represents a restricted subset of DSM-IV criteria for ADHD and does not recognize the DSM-IV predominantly inattentive subtype. The diagnosis of ADHD in children is based on clinical history and symptom reviews obtained from parents, teachers, and others who have significant interaction with the patient. The DSM-IV and the ICD-10 do not give guidelines for integrating information from multiple sources, which can be problematic if there is disagreement between parents, teachers and health professionals. Neither of these diagnostic algorithms provides explicit operational definitions of specific symptoms, and although the symptoms are not equal in their ability to predict diagnosis, they may be weighted equally in making diagnostic decisions. Accordingly, diagnosis is often subjective and without recourse to any reliable measures related to the neurobiological basis for the disorder. This is particularly concerning as accurate diagnosis is the key to effective management of ADHD and a false diagnosis may result in the medication of healthy individuals (including children), using psychoactive drugs.

fMRI is a neuroimaging technology which has been used in researching functional aspects of central nervous system disorders. fMRI is an application of nuclear magnetic resonance technology in which functional brain activity is detected usually in response to an activation task performed by a patient. fMRI is capable of detecting localized event-related brain activity and changes in this activity over time. Its principal advantages are its strong spatial and temporal resolution and, as no isotopes are used, a virtually unlimited number of scanning sessions that can be performed on a given subject, making within subject designs feasible. fMRI operates by detecting increases in cerebral blood volume that occur locally in association with increased neuronal activity. A widely used fMRI method for detecting brain activity is based upon the blood oxygenation level dependent (BOLD) response. The BOLD signal arises as a consequence of a ‘paradoxical’ increase in blood oxygenation, presumably due to increased local blood flow in excess of local metabolic demand and oxygen consumption following neuronal activity. An increase in blood oxygenation results in increased field homogeneity (increase in T2 and T2*), less dephasing of spins, and increased MR signal intensity on susceptibility-weighted MRI images.

No diagnostic system is currently available that can provide clues to the neurobiological basis of this disorder and reliable and quantifiable data relating to ADHD and its symptoms. However, fMRI has been under increasing development as an instrument for assessing neurobiological circuitry that underlies neurological disorders and for measuring the brain's response to therapeutic and especially pharmacological interventions.

### SUMMARY OF THE INVENTION

The present invention comprises a system for detecting neurological abnormalities related to Attention Deficit Hyperactivity Disorder (ADHD), diagnosing and assessing the severity of the disease and gauging the efficacy of therapies in treating the disorder. The system uses an MRI scanner to implement a functional magnetic resonance imaging (fMRI) scanning process in which a working memory and sustained attention task such as an N-Back task is performed by the patient during MRI scanning. The MRI scanner generates a time image series of MRI scan data showing functional activity in the brain generated in conjunction with the performance of the working memory and sustained attention task.

The working memory and sustained attention task is employed in order to stimulate activity in regions of the brain such as the left and right inferior frontal and inferior parietal network regions that are known to be directly affected by ADHD. In the preferred embodiment an N-Back task is used that involves the presentation of pseudorandom sequences of letters to participants who respond to the occurrence of letters previously signaled as target letters that are maintained in working memory. The N-Back task preferably includes four related procedures of parametrically increasing difficulty and memory load including zero-back, one-back, two-back and three-back conditions. The 0-back (“0B”) condition serves as a sensorimotor control task in which participants respond to a single pre-specified target letter and provides a baseline to which each of the three other working memory conditions can be compared. In the working memory conditions subjects respond to letters if they match target letters previously presented to them that are separated by specified intervals. For the one-back (“1B”) condition, subjects respond to a letter if it matches the letter that came immediately before the last letter. For 2B and 3B conditions, subjects respond if the current letter matches the letter presented 2 or 3 letters previous to it, respectively. The working memory and sustained attention task MRI data are analyzed by making comparisons between the data for the individual patient and standards for functional brain activity responsive to identity recognition tasks derived from reference data from healthy patients. On the basis of these comparisons symptoms related to ADHD may be detected and the presence and progress of the disorder in the patient may be diagnosed.

In a further embodiment a medication intended to address symptoms related to ADHD is administered to the patient. The resulting task-active MRI data from the patient are analyzed and compared with working memory and sustained attention task data elicited from the patient when not subject to the therapy. The patient's data may also be compared with reference data derived from a reference database including working memory and sustained attention task activity MRI data from a large number of healthy subjects and from subjects known to be afflicted with ADHD. The effectiveness of the medication can then be evaluated based on the comparative severity of the symptoms detected in said patient.

The fMRI time-series image data collected in conjunction with the performance of the N-Back activation tasks is analyzed to examine differences between the experimental conditions for different individuals and across different groups including controls. The analysis is focused on the left and right frontal operculum/insula as primary regions of interest (ROI) and on detection of hypoactivation under intermediate and high working loads.

It is an object of the present invention to provide a system for detecting neurological abnormalities associated with ADHD in an efficient, consistent and reliable manner using fMRI technology.

It is a further object of the present invention to provide a system for accurately diagnosing ADHD and assessing the severity of the disorder using fMRI technology.

It is another object of the present invention to provide an activation task adapted for use in fMRI studies and designed for stimulating brain activity in regions of the brain known to be affected by ADHD.

### BRIEF DESCRIPTION OF THE DRAWINGS

FIG. 1 provides a diagrammatic illustration of a magnetic resonance imaging machine and its major components as adapted for performing functional magnetic resonance imaging studies.

FIG. 2 provides a diagrammatic illustration of the MRI system components specifically dedicated to the performance of functional magnetic resonance imaging studies in accordance with the present invention.

FIG. 3 provides a flowchart illustrating the operative process for detecting the symptoms, diagnosing and determining the staging of ADHD in accordance with the present invention.

FIG. 4 provides a flowchart illustrating the operative process for detecting the symptoms and gauging the efficacy of medications intended to treat ADHD in accordance with the present invention.

FIG. 5 provides a graphical summary including brain images and graphs showing task active MRI results of normal and ADHD affected groups on and off the medication Methylphenidate (MP) using the N-Back task and analyzing the collected fMRI data in accordance with the present invention.

### DETAILED DESCRIPTION OF THE INVENTION

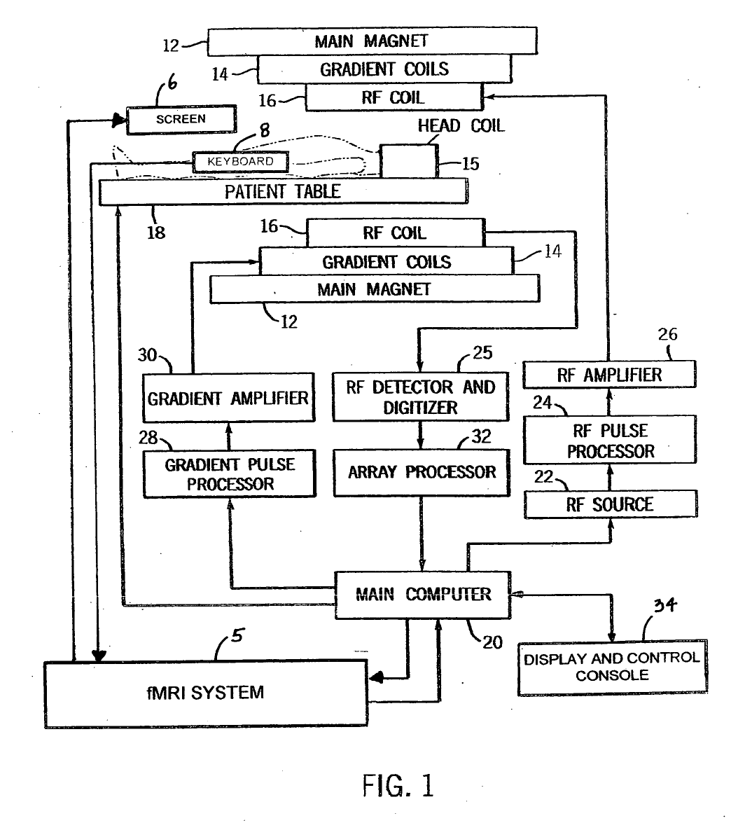

Referring now to FIG. 1, the basic components of a magnetic resonance imaging (MRI) machine 10 are shown including the fMRI system 5, which operates in conjunction with the MRI machine 10. A main magnet 12 produces a strong B₀ main magnetic field for use in the imaging procedure. Within the magnet 12 there are gradient coils 14 for producing a gradient in the B₀ field in the X, Y, and Z directions as necessary to provide frequency discrimination. A head coil 15 is also used to improve accuracy and resolution for studies involving the brain. Within the gradient coils 14 there is a radio frequency (RF) coil 16 for producing RF pulses and generating the B₁ transverse magnetic field necessary to rotate magnetic spins by 90° or 180°. The RF coil 16 also detects the return signal from the magnetic spins induced within the patient's body and supplies these signals to the RF detector and digitizer 25. The patient is positioned within the main magnet by a computer controlled patient table 18. The scan room is surrounded by an RF shield, which prevents the magnetic fields and high power RF pulses from radiating out through the hospital and prevents the various RF signals from television and radio stations from being detected by the imager. The heart of the imager is the main MRI computer 20 that controls the components of the imaging system. The RF components under control of the computer include the radio frequency source 22 and pulse programmer 24. The source 22 produces a sine wave of the desired frequency. The pulse programmer 24 shapes the RF pulses into apodized sync pulses. The RF amplifier 26 greatly increases the power of the RF pulses. The computer 20 also controls the gradient pulse programmer 28 which sets the shape and amplitude of each of the three gradient fields. The gradient amplifier 30 increases the power of the gradient pulses to a level sufficient to drive the gradient coils 14. In most systems an array processor 32 is also provided for rapidly performing two-dimensional Fourier transforms. The MRI computer 20 may then off-load Fourier transform tasks to this faster processing device. The operator of the imaging machine 10 provides input to the main MRI machine computer 20 through a display and control console 34. An imaging sequence is selected and customized by the operator from the console 34. The operator can see the MRI images on a video display located on the console 34. The fMRI system 5 controls the task display screen 6 visible to the subject and receives responses from the keyboard device 8 and coordinates the sequencing of activation task and MRI scanning procedures by exchanging signals with the main MRI computer 20.

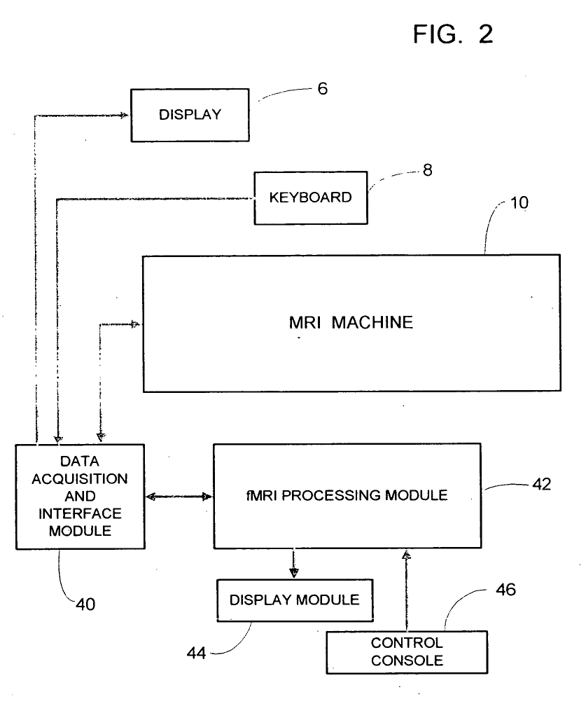

Referring now to FIG. 2, the fMRI system 5 includes the data acquisition and interface module 40, the processing module 42, the display module 44 and the input console 46 as well as the subject projection screen or display 6 and subject keyboard device 8. The module 40 directs the display of images to the subject on the screen 6 and also collects and preprocesses output responses from the subject provided from the keyboard device 8. The processing module 42 filters and analyses the fMRI data supplied to it by the data acquisition and interface module 40 by creating anatomical 3-dimensional datasets, converting the anatomical volumes into Talairach coordinate space, concatenating the functional time series datasets from multiple runs, registering the 3D time datasets to bring them into alignment, warping the functional datasets into Talairach coordinates, spatially blurring the images, performing deconvolution to compute the hemodynamic response to the stimuli, and calculating the change in hemodynamic response or BOLD contrast as a percent signal change over the region of interest (ROI). The processing module 44 also analyses the data and compares the data with normative data, indices and standards derived from a normative database of data acquired under comparable conditions from large numbers of healthy subjects and patients afflicted with the same CNS disorders. The display module 44 displays the results The visual stimuli for the activation tasks are computer-generated by the fMRI system 5 and rear-projected (video projector) on an opaque screen 6 located in the vicinity of the subject's feet. The subjects view the screen through prism glasses attached to the head coil 15. Corrective lenses can be provided if necessary. The viewing distance is usually about 220 cm. A non-ferrous three-button key-press (keyboard) device 8 made from force-sensing resistors is preferably used to record responses, accuracy and reaction time. To provide precise time synchronization between the presentation of visual stimuli and the scan sequence, a trigger signal coincident with the acquisition of each MR image is fed into the computer controlled video display 6 by the fMRI system 5.

A General Electric Signa EXCITE 3.0 Tesla MRI scanner is used for implementing the present invention although any of a number of commercial MRI scanners having 3.0 or 1.5 Tesla fields could be used. Typical imaging parameters involve, for example, the acquisition of 36 contiguous axial slices that cover the entire brain (typically 4 mm thick) using a blipped gradient-echo, echoplanar pulse sequence (echo time (TE)=25 msec; interscan period (TR)=300 msec; field of view (FOV)=24 cm; 64×64 voxel matrix; 3.75 mm.×3.75 mm in-plane resolution). High resolution (124 axial slices) spoiled GRASS (gradient-recalled at steady-state) anatomic images [TE=3.9 ms; TR (repetition time)=9.5 ms, 120 flip angle, NEX (number of excitations)=1, slice thickness=1.0 mm, FOV=24 cm, matrix size=256×224] are acquired prior to functional imaging for anatomical localization of functional activation and co-registration. Stimulus presentation and general communication to the patient in the MR scanner is accomplished with stereo audio headphones and computer generated images fed into a digital LCD projector which are back projected to the subject and viewed by the patient through prismatic glasses. Subject responses are recorded on a small hand held keyboard or response device including multiple response buttons. Response data, including task responses, accuracy, and reaction time (RT), are acquired on a PC for off-line analysis.

Foam padding or a vacuum bead system that molds around the patient's head is preferably used to limit head motion within the head coil. Head movement, typically subvoxel (<2 mm), is viewed in cine format. The image time series is spatially registered to minimize the effects of head motion and a 3D volume registration algorithm may be used align each volume in each time series to a fiducial volume through a gradient descent in a nonlinear least squares estimation of six movement parameters (3 shifts, 3 angles) and is designed to be efficient at correcting motions of up to a few mm and rotations up to a few degrees. Excessive head movement beyond what can be accurately corrected may entail elimination of participants.

Subjects effected by ADHD are known to exhibit unique activation patterns involving the inferior frontal and inferior parietal regions of the brain and more particularly hypoactivation in the frontal operculum/insula areas (BA 13/45; left insula: −33,17,4; right insula 34,15,5). Under the influence of working memory and sustained attention tasks such as N-Back tasks, ADHD groups demonstrate significant differences in activation intensity compared to control groups with such impairments tending to indicate ADHD and tracking the clinical course of the disorder. Accordingly, fMRI based measures of working memory and sustained attention responsive to N-Back tasks and focused on these regions of interest (parietal and bilateral inferior frontal) can act as a sensitive marker to enable the detection of neurological abnormalities associated with ADHD and the tracking of the course of abnormalities associated with the disorder.

The generalized N-Back task consists of pseudorandom sequences of letters presented to participants who respond to the occurrence of pre-specified target letters previously committed to memory. The N-Back task is a parametrically designed so that working memory load can be incrementally varied. The stimuli consist of pseudorandom sequences of consonants visually presented in lower or uppercase form. Each stimulus is centrally presented in black on a white background for a duration of 500 ms with an interstimulus interval of 2500 ms. The N-Back task preferably includes four blocked conditions comprising 0-Back, 1-Back, 2-Back and 3-Back conditions. The 0-Back (“0B”) condition serves as a sensorimotor control task in which participants respond to a single pre-specified target letter. The 0B condition provides a baseline to which each of the three working memory conditions can be compared. In the working memory conditions subjects respond to letters if they match target letters previously presented to them and separated by specified intervals. For the 1-back (“1B”) condition, subjects respond to a letter if it matches the letter that came immediately before the last letter. For 2B and 3B conditions, subjects responded if the current letter matches a letter presented 2 or 3 letters previous to it, respectively. The 0B condition always alternates with the working memory conditions (1B, 2B, and 3B). Task instructions are indicated by presentation of a written display such as “1-Back”. Each of the experimental conditions consists of fifteen consonants, five of which are targets. Participants are administered three runs of the four experimental conditions arranged in a random order. Accordingly, the 0B, 1B, 2B, and 3B conditions are each administered six times, 2 times per run. Condition order is randomized such that each condition is presented once, followed by a rest period of 12 seconds and then a second randomized cycle of each condition is presented. Each run begins and ends with 12 seconds rest. Participants briefly practice the task prior to actual scanning.

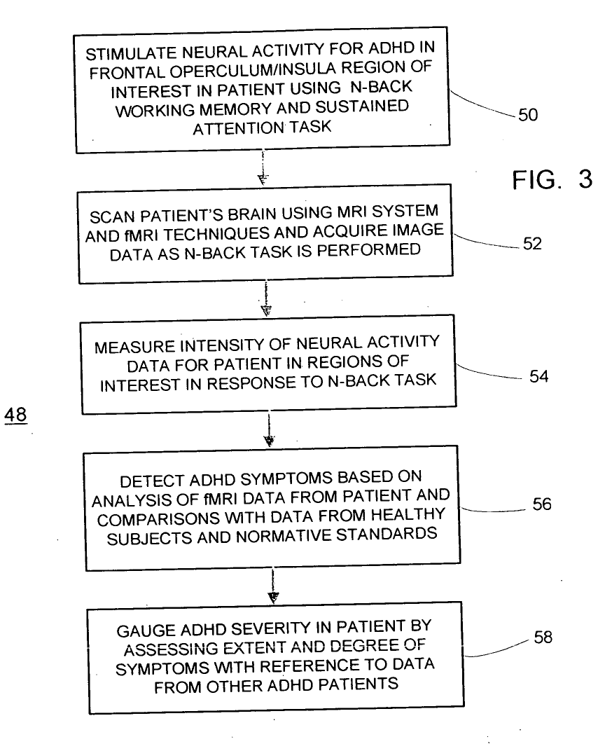

Referring now to FIG. 3, the operative process 48 for detecting the symptoms, diagnosing and determining the staging of ADHD includes the steps 50, 52, 54, 56 and 58. In step 50 the patient is prompted using an N-Back working memory and sustained attention task in order to generate neural activity in those regions of interest in the patient's brain that may be affected by ADHD such as the frontal operculum/insula. The N-Back task includes zero-back, one-back, two-back and three-back conditions. Step 52 is performed concurrently with step 50 so that scanning and data acquisition by the MRI machine both take place as brain activity is stimulated in response to the N-Back task. In step 52 N-Back related MRI data indicative of the functional MRI brain activity of the patient responsive to the N-Back task is acquired and recorded by the MRI scanning system. The N-Back related MRI data is then analyzed in step 54 and the intensity of neural activity in the region of interest in response to the task is measured. Thereafter, in step 56 ADHD symptoms are detected by making comparisons between the patient's N-Back related data, or indices derived from these data, and reference data, reference indices, or normative standards for functional brain activity responsive to N-Back tasks as derived from MRI data from healthy subjects. If symptoms characteristic of ADHD are detected in the patient then in step 58 the severity of the patient's condition is estimated by analyzing the neural activation intensity in the frontal operculum/insula regions of interest in response to the N-Back task and assessing the extent and degree of the patient's symptoms in comparison with similar fMRI data from other ADHD patients. Accordingly, the patient may be diagnosed as having or not having the ADHD based on the symptoms detected using fMRI and if the patient is in fact diagnosed with the disease the severity may be determined in step 52 based on assessing extent and degree of said symptoms.

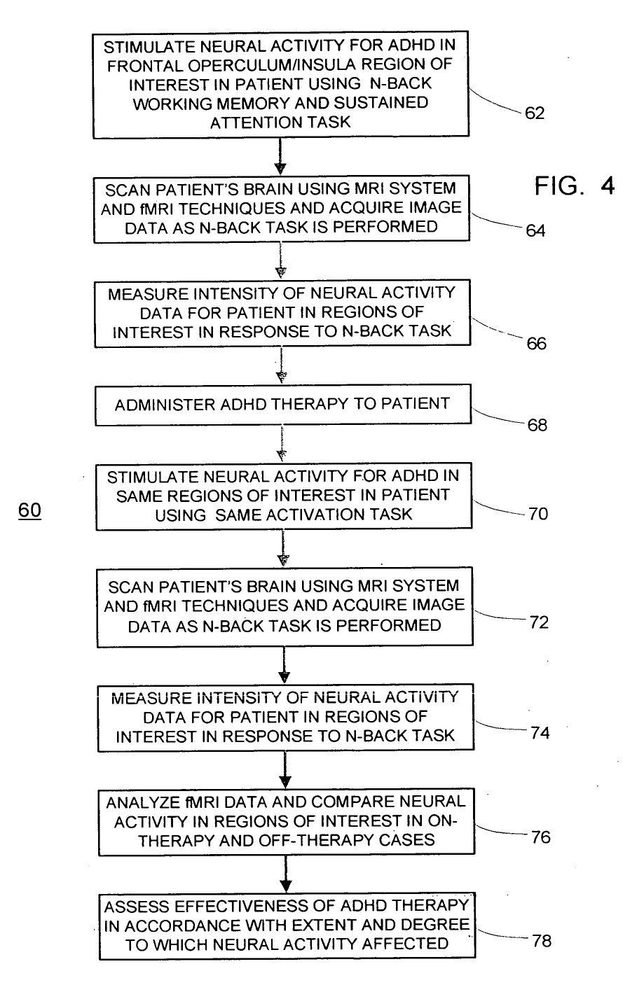

Referring now to FIG. 4, the operative process 60 for detecting the symptoms and gauging the efficacy of medications intended to treat ADHD includes the steps 62, 64, 66, 68, 70, 72, 74, 76 and 78. Steps 62, 64, and 66 are similar to steps 50, 52 and 54 as described above and involve activating a selected region of the brain using an N-Back type task, concurrently acquiring task-active MRI data responsive to the N-Back task, and measuring the intensity of the patient's neural activity in the regions of interest. However, in step 70 a therapy or medication intended to treat ADHD is administered to the patient. Steps 72, 74 and 76 are again similar to steps 50, 52 and 54 as described above and involve activating a selected region of the brain using an N-Back type task, concurrently acquiring task-active MRI data responsive to the N-Back task and measuring the intensity of the patient's neural activity in the regions of interest. However, in step 78 the effectiveness of the therapy or medication administered in step 70 is assessed based on the comparative levels of neural activity achieved in the operculum/insula regions of interest of the patient in response to the N-Back task and the relative severity of the symptoms detected in the patient when on and off therapy or when under the influence of medication and when not.

The imaging analysis consists of a comparison of the signal intensity and the spatial extent of regional cerebral activity arising with respect to the N-Back working memory and sustained attention activation task. Region of Interest (ROI) analyses are focused on inferior frontal and inferior parietal network regions of the brain.

Several publicly available software programs such as AFNI (Medical College of Wisconsin in Milwaukee, Wis.) and BrainVoyager (Brain Innovation B.V. in Maastricht, Netherlands) have been developed that allow for whole-brain, 3D fMRI activation mapping and within- and between-subjects statistical comparisons and also include extensive statistical routines. Typically, all whole-brain fMRI data are converted to 4D data sets (time plus 3 spatial dimensions). Image time series are spatially registered to minimize effects of head motion. The runs are then concatenated in order to obtain a single time-series per subject. Multiple regression is used to analyze individual time series data for each participant. Parameters in this analysis include a baseline (rest), a linear trend, and boxcar regressors for each of the blocked N-Back experimental conditions (0B 1B, 2B, and 3B). These analyses can test the degree to which the multiple regression model predicts individual image values under each of the separate experimental conditions on a voxel-wise basis. Functional imaging data are converted to Talairach stereotactic coordinate space (1 mm³ voxels) and typically blurred using a 6 mm Gaussian full-width half-maximum (FWHM) filter to compensate for intersubject variability in anatomic and functional anatomy. Functional images are generated using t-tests which examine separately differences between each of the four experimental conditions versus rest.

While voxel-wise statistical analyses are easy to implement, they may distort information due to normal variations in cortical and subcortical topography. These differences may become magnified when comparing brain activation patterns across groups of subjects (healthy vs. severe ADHD vs. mild ADHD). In the preferred embodiment there are several regions and subregions of the brain that comprise specific regions of interest (ROIs) to be analyzed in greater detail. An activated region may be defined by an individual voxel probability of p<0.0001 for both control subjects and ADHD participants (t>5.62, df=15), with a minimum cluster size threshold of 200 μl. Regions of interest (ROIs) are defined by creating a common activation map that included regions activated by all working memory conditions (relative to rest) for both subject groups. The mean percent signal change will be calculated for each group within each region. Statistical comparisons of the functional imaging maps will be generated by performing a 2 (patient vs control)×3 (Drug condition)×3 (N-Back condition) mixed model ANOVA. As a part of the overall analyses two dependent values are calculated for each such region of interest (ROI): (1) the number of activated voxels divided by the total number of voxels in the region, a measure of the spatial extent of the activated region, and (2) the mean % area-under-the-curve (% AUC) of the activated voxels, a measure of the intensity of the activated region.

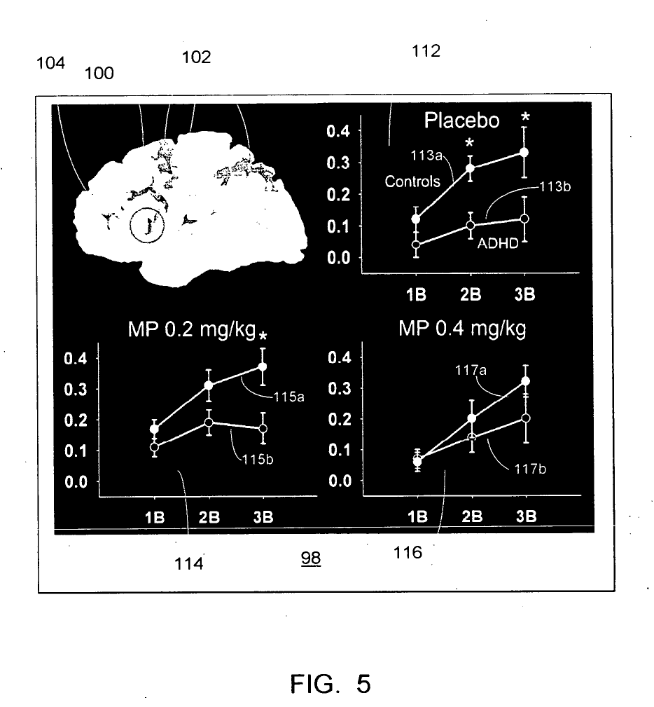

Referring now to FIG. 5, the graphs and brain image 98 show exemplary data for controls and for participants previously identified as having ADHD pursuant to behavioral studies. The data for the graphs and brain image 98 were developed pursuant to the performance of N-Back activation tasks. Graphs 112, 114 and 116 illustrate differences in fMRI signal intensity in specifically identified regions of interest between the ADHD and control groups. As shown in brain image 100 and consistent with previous functional imaging studies, ADHD and control subjects activated the bilateral premotor (BA 6/8), pre-supplementary motor area (preSMA; BA 6), right dorsolateral prefrontal, and bilateral inferior parietal (BA 40) cortices, as well as the basal ganglia, thalamus, and bilateral cerebellum although no significant between group differences in activation intensity were demonstrated in these regions. However, Region of Interest (ROI) analysis identified two regions demonstrating a significant between-group difference in activation intensity: the left and right frontal operculum/insula (BA 13/45-Talairach stereotactic coordinates) highlighted within circle 104 in brain image 100. Darker brain regions 102 in image 100 represent areas commonly activated by the N-Back task (collapsing across N-Back condition and group) during placebo imaging sessions. The highlighted region within circle 104 demonstrates significant differences in MR signal intensity between ADHD subjects and controls.

The graphs 112, 114 and 116 illustrate differences in fMRI signal intensity in the left insula/frontal operculum as highlighted at 104 between ADHD effected and control groups under placebo and different MP treatment dosages across the 1-B, 2-B and 3-B activation task conditions. Group differences in brain activation observed during the placebo condition tended to disappear when subjects were treated with MP. The plots 113a and 113b, 115a and 115b and 117a and 117b within graphs 102, 104 and 106 show changes in percent MR signal intensity as a function of group and N-Back condition, *p<0.05 for placebo, 0.2 mg/kg and 0.4 mg/kg MP dosage conditions. Plots 113a, 115a and 117a represent the control group and plots 113b, 115b and 117b represent the ADHD group and illustrate the detection of significant differences in brain function at the regions of interest (left frontal operculum/insula) indicative of the symptoms of ADHD.

Although the invention has been described with reference to certain embodiments for which many implementation details have been described, it should be recognized that there are other embodiments within the spirit and scope of the claims and the invention is not intended to be limited by the details described with respect to the embodiments specifically disclosed. For example, semantic retrieval activity may be invoked by other working memory and sustained attention tasks or other combinations of the N-Back activation conditions.

1. A method of assessing neurological abnormalities associated with deficits in working memory and sustained attention that are characteristic of ADHD, comprising the steps of:
a) scanning a patient's central nervous system using fMRI techniques;
b) stimulating neurological activity in the inferior frontal and inferior parietal network regions of the patient's central nervous system by having the patient perform a series of activation tasks having different working memory and sustained attention loads;
c) measuring the level of intensity of neural activity in said regions which are attained under the influence of said tasks; and
d) comparing said level of intensity in said regions of said patient's central nervous system with normative standards based on levels of intensity of neural activity in the same regions of the central nervous system measured in similar individuals not effected by ADHD scanned using similar fMRI techniques who performed a similar series of working memory and sustained attention tasks.

2. The method of claim 1, wherein:
said working memory and sustained attention tasks comprise N-Back working memory and sustained attention activation tasks, and
said network regions include the frontal operculum/insula regions.

3. The method of claim 1, further including the step of:
e) tracking the accuracy with which the patient completes a control condition in order to verify that the patient is fully engaged in performing said activation tasks.

4. The method of claim 2, wherein said step of stimulating neurological activity includes the step of:
parametrically increasing the working memory and sustained attention load on the patient by changing said tasks from 0-Back 1-Back to 2-Back to 3-Back tasks.

5. The method of claim 2, wherein said N-Back working memory and sustained attention activation tasks include the sub-steps of:
i) identifying target symbols to the patient for response by the patient when these symbols are repeated in a specified pattern,
ii) visually presenting a series of symbols to the patient including the target symbols repeated in patterns including the specified pattern, and
iii) having the patient respond when the target symbols occur within the specified pattern.

6. The method of claim 5, wherein:
said pattern comprises target symbols repeated as second occurring symbols following initial occurrences of such symbols.

7. The method of claim 5, wherein:
said symbols comprise consonants.

8. The method of claim 2, wherein:
said working memory and sustained attention tasks comprise a plurality of different N-Back conditions having different activation loads.

9. The method of claim 8, wherein said N-Back tasks include the sub-steps of:
i) identifying target symbols to the patient for response by the patient when these symbols are repeated in a specified pattern,
ii) visually presenting a series of symbols to the patient including the target symbols repeated in patterns including the specified pattern, and
iii) having the patient respond when the target symbols occur in the specified pattern.

10. A process adapted for assessing neurological abnormalities associated with ADHD for use in conjunction with fMRI scanning, said process comprising the steps of:
a) stimulating neural activity in the frontal operculum/insula regions of the brain of a patient suspected of having ADHD by having said patient perform an N-Back working memory and sustained attention task;
b) acquiring and recording a first set of fMRI data indicative of the functional brain activity of the patient responsive to said N-Back working memory and sustained attention task by scanning the patient's brain using an MRI scanner in conjunction with having him perform said N-Back task; and
c) detecting a neurological abnormalities symptomatic of ADHD in said patient analyzing said fMRI data and comparing the intensity level for neural activity in said regions indicated by said data for said patient with standards for intensity levels for neural activity defined by normative data for neural activity in healthy individuals with respect to said regions based on fMRI data acquired from healthy subjects when responding to a similar working memory and sustained attention task.

11. The process of claim 10, wherein:
said N-Back working memory and sustained attention activation task includes 0-Back, 1-Back, and 2-Back conditions.

12. The process of claim 10, further including the step of:
d) tracking the accuracy with which the patient completes said working memory and sustained attention tasks using a 0-Back control condition in order to verify that the patient is fully engaged in performing said activation tasks.

13. The process of claim 10, wherein said step of stimulating central nervous system regions includes the sub-step of:
parametrically increasing the working memory and sustained attention load on the patient by changing said tasks from 1-Back to 2-Back to 3-Back.

14. The process of claim 10, wherein said N-Back activation tasks include the sub-steps of:
i) identifying target symbols to the patient for response by the patient when these symbols are repeated in a specified pattern,
ii) visually presenting a series of symbols to the patient including the target symbols repeated in patterns including the specified pattern, and
iii) having the patient respond when the target symbols occur in the specified pattern.

15. The process of claim 14, wherein:
said specified pattern comprises target symbols repeated as second occurring symbols following initial occurrences of such symbols, and said symbols comprise consonants.

16. The process of claim 10, further including the steps of:
e) assessing the severity of neurological abnormalities related to ADHD in said patient by making comparisons between said data for said patient and normative data defining standards for the intensity of functional brain activity in said regions based on fMRI data acquired from patients known to be afflicted with ADHD with differing degrees of severity.

17. The process of claim 10, further including the steps of:
e) administering a therapy to said patient intended to address neurological symptoms related to ADHD;
f) stimulating neural activity in said regions of the brain of said patient by having the patient perform said task while under the influence of said therapy;
g) acquiring and recording a second set of fMRI data indicative of the functional MRI brain activity of the patient responsive to said task by scanning the patient's brain using an MRI scanner in conjunction with having him perform said N-Back task while under the influence of said therapy; and
h) gauging the effectiveness of said therapy by comparing the second set of fMRI data acquired while said patient is under the influence of said therapy with the first set of fMRI data acquired while said patient is not under the influence of said therapy.

18. The process of claim 17, in which:
said step of administering a therapy comprises administering a pharmaceutical medication to the patient.

19. The process of claim 17, wherein:
said N-Back working memory and sustained attention task comprises 0-Back, 1-Back, 2-Back and 3-Back conditions.

20. The process of claim 17, wherein said N-Back task include the sub-steps of:
i) identifying target symbols to the patient for response by the patient when they are repeated in a specified pattern,
ii) visually presenting a series of symbols to the patient including the target symbols repeated in the specified pattern, and
iii) having the patient respond when the target symbols occur in the specified pattern.

21. The process of claim 20, wherein:
said specified pattern comprises target symbols repeated as second occurring symbols following initial occurrence of such symbols, and
said symbols comprise alphanumeric characters.

22. A process based on the use of fMRI techniques which is adapted for assessing neurological abnormalities associated with ADHD and gauging the effectiveness of a therapy intended to address ADHD symptoms, said process comprising the steps of:
a) stimulating neurological activity in the inferior frontal and inferior parietal network regions of the central nervous system of a patient suspected of having ADHD by having said patient perform a working memory and sustained attention task;
b) acquiring and recording a first set of fMRI data indicative of the functional brain activity of the patient responsive to said working memory and sustained attention task;
c) administering a therapy to said patient intended to address neurological symptoms related to ADHD;
d) stimulating neurological activity in the inferior frontal and inferior parietal network regions of the central nervous system of said patient by having the patient perform said working memory and sustained attention task while under the influence of said therapy;
e) acquiring and recording a second set of fMRI data indicative of the functional MRI brain activity of the patient responsive to said working memory and sustained attention task while under the influence of said therapy;
f) comparing the second set of fMRI data acquired while said patient is under the influence of said therapy with the first set of fMRI data acquired while said patient is not under the influence of said therapy; and
g) gauging the effectiveness of said therapy based on the results of comparing said sets of fMRI data.

23. The process of claim 22, in which:
said inferior frontal regions comprise the frontal operculum/insula regions, and
said step of administering a therapy to said patient comprises administering a pharmaceutical medication to the patient.

24. The process of claim 22, wherein:
said working memory and sustained attention task comprises an N-Back task having a plurality of different N-Back activation conditions having different activation loads.

25. The process of claim 24, further including the step of:
tracking the accuracy with which the patient completes a control condition in order to verify that the patient is fully engaged in performing said activation task.

26. The method of claim 24, wherein:
said steps of stimulating neurological activity include the sub-step of:
parametrically increasing the working memory and sustained attention load on the patient by changing task conditions from 1-Back to 2-Back to 3-Back conditions.
